(19) United States
(12) Patent Application Publication
(10) Pub. No.: US 2013/0022650 A1
(43) Pub. Date: Jan. 24, 2013

(54) COMPOSITIONS CONTAINING SALMETEROL, FLUTICASONE AND CROMOGLICIC ACID

(71) Applicant: Mahmut Bilgic, Istanbul (TR)

(72) Inventor: Mahmut Bilgic, Istanbul (TR)

(21) Appl. No.: 13/519,018

(22) PCT Filed: Dec. 20, 2010
(86) PCT No.: PCT/TR2010/000250

§ 371 Date: Oct. 1, 2012

(30) Foreign Application Priority Data
Dec. 25, 2009 (TR) 200909791

(51) Int. Cl. A61K 31/56; A61K 9/14; A61P 11/00

(52) U.S. Cl. 424/400; 514/171

(57) ABSTRACT

The present invention relates to the use of cromoglicic acid and/or nedocromil salts in pharmaceutically effective amounts in a pharmaceutical composition containing salmeterol or a pharmaceutically acceptable salt thereof as a 32-agonisti combined with fluticasone or a pharmaceutically acceptable salt thereof as a corticosteroid for the treatment of the respiratory diseases.

The present invention is related to a pharmaceutical composition containing the combination of salmeterol or a pharmaceutically acceptable salt thereof as a β₂-agonist, and fluticasone or a pharmaceutically acceptable salt thereof as a corticosteroid and a pharmaceutically effective amount of cromoglicic acid and/or nedocromil salts is used for the treatment of respiratory disorders.

In pharmacology, the molecules, which are known as β₂-agonists, which are classified into two groups i.e. short acting and long acting, cause relaxation of smooth muscle around the airways that results in dilation of bronchial passages. Due to this characteristic, these drugs are used for the treatment of asthma and chronic obstructive pulmonary disorder (COPD).

In 2005 US Food and Drug Administration reported that most of the long acting β₂-agonists increase wheezing symptoms in patients. After that, in a study conducted by Cornell and Stanford Universities in 2006, it was concluded that long term administration of beta-agonists and/or administration of a high dose of beta-agonists for the treatment of COPD increase health problems in the airways. Thus, different methods should be developed for the administration of β₂-agonists for the treatment of asthma and COPD.

Another group of drugs used in the treatment of asthma and COPD are corticosteroids. These molecules, which have been used approximately since 1950s, lead to reduction of mucus secretion by suppressing inflammation that is origin of the asthma, thus problems of asthma patients are resolved. When this group of drugs is administered systemically, they cause serious side effects such as osteoporosis, high cholesterol, edema, headache, weight gain, sleep disorders, various skin complications and growth retardation in children. Hence, the delivery methods by which the medicament enters the systemic circulation in an amount as less as possible, is preferred. These drugs are preferred to be administered by inhalation route for the treatment of asthma in order to decrease the side effects and to deliver the dose of the drug as high as possible to the lungs.

As mentioned above, β₂-agonist drugs can induce the respiratory disorders when they are used individually and at high doses. The fact that these drugs which are inevitably used for the treatment of asthma due to their strong efficacy, is combined with different drugs which are efficient on treatment of the same disorder, provides advantages which are reduction of the amount of β₂-agonist to be administered that cause alleviation and/or disappearance of side effects, and a synergistic effect provided by such a combination of two different substances. For this purpose, one of the combination products, that has been used for many years is salmeterol xinafoate and fluticasone propionate.

Ideally, delivery methods which provide the drug to be delivered directly and rapidly to the target area and hence to exhibit the desired effect at lower doses and as a result cause alleviation and/or disappearance of side effects, are preferred. Considering the serious side effects of corticosteroids resulting from the systemic administration of these drugs, the importance of the efficient delivery of this combination by inhalation route is realized. One of the most common issues in the prior art is development of these delivery methods and the suitable devices for such methods.

Generally, three main different methods and corresponding inhalers are used for delivery of medicaments by inhalation route. These are; (a) nebulization device, (b) metered dose inhalers and (c) dry powder inhalers. Dry powder inhalers come into prominence due to their ease of use. However, the delivery of the effective amount of the dry powder formulation to the lungs is still a problem and the development of the new formulations in order to solve this problem is still needed.

Lactose is used as a carrier in most of the dry powder formulations such as the combination of salmeterol xinafoate and fluticasone propionate. The amount of lactose used in a single dose is usually in the range of 10 mg to 25 mg, which is about 500-2500 times more than the amount of the active agent. However, a large amount of lactose used as a carrier in dry powder formulation causes side effects such as coughing, throat irritation, etc. Furthermore, use of dry powder formulations containing large amount of lactose by patients with allergy and/or lactose intolerance (sensivity to high-dose intake of lactose), causes symptoms such as nausea, stomach cramps, overfullness of the stomach, swelling of stomach, flatulence, diarrhea and hives plaque due to the fact that most of the drugs administered by inhalation are swallowed. Dry powder formulations containing a β₂-agonists and a corticosteroid need to be developed to overcome these drawbacks mentioned above.

Salts of cromoglicic acid and nedocromil are used for the treatment of bronchospasm in pharmacology. Because of this property, it is used for the treatment of asthma, allergic rhinitis, allergic and vernal conjunctivitis diseases.

There are products which are marketed by the trademarks of “Intal” and “Cromohexal” provide up to 20 mg sodium cromoglycate per dose.

The present invention relates to use of salts of cromoglicic acid and/or nedocromil in a pharmaceutical composition comprising the combination of salmeterol or a pharmaceutically acceptable salt and fluticasone or a pharmaceutically acceptable salt. Initially, for the purpose of reducing the amount of lactose and hence alleviation of side effects caused by lactose in dry powder formulations comprising salmeterol and fluticasone, a salt of cromoglicic acid and/or nedocromil has been added to the formulation. But surprisingly, it has been found that when the amount of a salt of cromoglicic acid and/or nedocromil is increased and it is used in effective amounts in the dry powder formulation containing salmeterol and fluticasone, the combination shows a synergistic effect and an unexpected benefit occurs for treatment of respiratory diseases. Therefore, according to the present invention, when a salt of cromoglicic acid and/or nedocromil are used in dry powder formulations containing salmeterol or a pharmaceutically acceptable salt thereof combined with fluticasone or a pharmaceutically acceptable salt thereof, the required amount of carrier is reduced and hence the side-effects caused by the carrier are reduced, and also an unexpected therapeutic effect is achieved for the treatment of respiratory diseases.

The present invention provides a medicament composition containing salmeterol or a pharmaceutically acceptable salt thereof and fluticasone or a pharmaceutically acceptable salt that are combined with a salt of cromoglicic acid and/or nedocromil in an effective amount, used in the treatment of respiratory diseases.

The medicament composition in accordance with the present invention optionally contains a carrier in addition to the medicaments mentioned above.

In another aspect, the invention provides use of salmeterol, fluticasone or a pharmaceutically acceptable salt thereof and a salt of cromoglicic acid and/or nedocromil in effective amounts for the preparation of a medicament which is used for the treatment of respiratory diseases particularly when an allergic case exists.

In another aspect, the present invention provides a method for the preparation for said medicament composition.

In an embodiment of the invention, salmeterol contained in the medicament composition may be in a form that is selected from a group comprising pharmaceutically acceptable salts and/or hydrates, and/or free base and/or polymorphs, amorphous forms, crystal forms and/or esters and/or solvates thereof. Preferably, salmeterol xinafoate is used.

In an embodiment of the invention, fluticasone contained in the medicament composition may be in a form that is selected from a group comprising pharmaceutically acceptable salts and/or hydrates, and/or free base and/or polymorphs, amorphous forms, crystal forms and/or esters and/or solvates, thereof Preferably, fluticasone propionate is used.

In an embodiment of the invention, a salt of cromoglicic acid and/or nedocromil can be selected from pharmaceutically acceptable salts thereof Preferably, it can be sodium cromoglycate and/or nedocromil sodium.

A medicament composition in accordance with the present invention that is in dry powder form, is administered via inhalation route optionally contains pharmaceutically acceptable carrier. The carrier can be selected from a group comprising arabinose, glucose, fructose, ribose, mannose, sucrose, trehalose, lactose, maltose, starches, dextran, sugar alcohols such as mannitol and saccharides.

Salmeterol is present in the amount of 5 to 100 μg, preferably 5 to 80 μg for a single dose of the medicament composition in accordance with the present invention.

Fluticasone is present in the amount of 5 to 1000 μg, preferably 5 to 700 μg for a single dose of the medicament composition in accordance with the present invention.

A salt of cromoglicic acid or nedocromil is present in the amount of 1 to 50 mg, preferably 1 to 25 mg for a single dose of the medicament composition in accordance with the present invention.

The ratio of the combination of β₂-agonist and corticosteroids to the salt of cromoglicic acid or nedocromil is preferably in the range of between 1:5 and 1:55000 in the medicament composition in accordance with the present invention. Additionally, a pharmaceutically acceptable carrier can be used in the medicament composition for the purpose of adjusting the amount of each dose of said medicament composition in dry powder form to the range of between 2 and 50 mg.

The medicament composition in accordance with the present invention is in the form of micronized dry powder particles. The active substances present in said medicament composition have average particle size in the range of 1 to 20 μm, preferably in the range of 1 to 6 μm. The carrier present in said medicament composition typically has average particle size of not more than 300 μm, preferably not more than 210 μm. The salt of cromoglicic acid and/or nedocromil present in said medicament composition as both an active substance and as an excipient, has average particle size in the range of 1 to 5 μm and/or in the range of 10 to 300 μm.

The inventors has encountered a problem about the failure in the delivery of the medicament composition in accordance with the present invention comprising combination of salmeterol and fluticasone with the salt of cromoglicic acid and/or nedocromil to the lungs in effective amounts during inhalation of said medicament composition. Various methods and devices have been developed in order to overcome this problem.

In the inhalation of the medicament composition in accordance with the present invention, use of a blister strip is preferred because of the fact that said blister strip provides ease of use by carrying multiple doses, it is reliable in terms of hygiene and it eliminates the moisture absorption of the medicament composition in dry powder form and it also provides the medicament composition to be administered in an accurate dose in each inhalation.

One of the main problems of the prior art is the effective delivery of the dry powder formulations used for the treatment of the respiratory disease to the lung. Each blister in the blister strip is opened by piercing with a piercing means or by peeling the lid sheet apart from the base sheet and then the medicament composition is ready for inhalation.

Dry powder inhalers, in which the blisters of the blister strip are opened by piercing, require additional mechanical components. Due to this, the size, the volume and the complexity of the inhalers are increased and this results in difficulties in use of the inhaler by patient. Moreover, some of the uninhaled medicament composition in dry powder form remains in the blister that is opened by penetrating, because of the roughnesses occurred during the penetration of the blister. Consequently, rate of utilization from the medicament composition in the blister reduces. Also small pieces, which ruptured from the blister during the penetration of the blister, can be inhaled with the medicament composition to the lungs during inhalation of the medicament.

Accordingly, another embodiment of the invention includes that the medicament composition in dry powder form containing the combination of salmeterol and fluticasone with the salt of cromoglicic acid and/or nedocromil, is stored in a blister strip that is opened by peeling, and it is administered by a dry powder inhaler.

The peelable blister strip which is used within the present invention is composed of lid sheet and base sheet. The base sheet is provided with cavities containing the medicament composition in dry powder form. Lid sheet, which covers the cavities containing the medicament composition in dry powder form, is peeled apart to make the composition ready for inhalation.

In another aspect, the present invention provides a method for administration of the medicament composition containing the combination of salmeterol and fluticasone with the salt of cromoglicic acid and/or nedocromil in effective amounts and optionally a pharmaceutically acceptable carrier, that is used by the patients, especially by the patient having an allergy to lactose, troubled with the respiratory disorders, wherein said medicament composition is stored in a blister strip to be administered with a dry powder inhaler.

The medicament composition in accordance with the present invention is in the form of micronized dry powder particles. The active substances present in said medicament composition have average particle size in the range of 1 to 20 μm, preferably in the range of 1 to 6 μm. The carrier present in said medicament composition typically has average particle size of not more than 300 μm, preferably not more than 210 μm. The salt of cromoglicic acid and/or nedocromil present in said medicament composition as both an active substance and as an excipient, has average particle size in the range of 1 to 5 μm and/or in the range of 10 to 300 μm.

The cavity volume of each blister in the peelable blister strip present in the dry powder inhaler, which is used for delivery of said medicament composition to the lung, is in the range of 20 to 30 mm³, preferably in the range of 21 to 25 mm³, most preferably in the range of 22 to 23 mm³.

The lid sheet and the base sheet which constitutes said blister strip are closed very tightly by using suitable method to provide impermeability.

According to the present invention, the lid sheet or the base sheet of the peelable blister strip consists of three layers. Two of these layers are polymeric layers and the other one is aluminium foil. Aluminium foil is conventionally used in both the lid sheet and the base sheet of the peelable blister strip to provide high humidity and gas protection, so that aluminum foil is used both in the lid sheet and in the base sheet of the peelable blister strip for the same purpose. These layers must have the sufficient thickness which provides the protection for the stability of humidity sensitive dry powder composition which is stored in blister cavity. Because of this reason, the thickness of aluminium foil that is used in the lid sheet and the base sheet of the peelable blister strip is in the range of 10 to 40 μm, preferably of 15 to 30 μm.

The other layers contained by the lid sheet and the base sheet of the peelable blister strip according to the present invention are polymeric layers. These polymeric layers may be made of either same or different polymers. The thickness of these polymeric layers depends on the type and properties of the polymeric substance used. Therefore, the thickness of each polymeric layer, which is used in the lid sheet and the base sheet of said blister strip, is in the range of 15 to 60 μm, preferably of 20 to 35 μm depending on the type of polymer used.

The inside layer of blister cavity of said blister strip which is in contact with dry powder formulation is polymeric layer because of the fact that aluminium foil causes adhesion of a part of the dry powder composition to inside layer of the cavity due to electrostatic forces, and hence cause uncontrolled dosing.

According to the present invention, the polymers used for forming polymeric layers are preferably selected from a group comprising thermo-plastic polymers such as polyethylene, polypropylene, polystyrene, polyolefin, polyamide, polyvinyl chloride, polyurethane.

The layers which are used for making up the lid sheet of said blister strip in accordance with present invention are preferably same with the layers used for making up the base sheet of the same; however the polymeric substances used for forming each polymeric layer are preferably different from each other. Moreover, each of the blister cavities which constitute the peelable blister strip can be different in shape providing that they have the properties defined above.

The inhaler used to inhale the dry powder composition in accordance with the present invention may be multi-dose inhalers present in the prior art. For this reason, in one aspect, the invention provides a medicament composition as mentioned before and in another aspect, the invention provides a method for delivery of said medicament composition to patient's lungs effectively as mentioned before.

The medicament composition in accordance with the present invention containing a β₂-agonist, a corticosteroid and a salt of cromoglicic acid and/or nedocromil, is stored in the peelable blister strip mentioned above and the amount of 5 to 50 miligrams of said medicament composition is inhaled by a multi-dose inhaler in each actuation of the inhaler.

The method for preparation of the medicament composition in accordance with the present invention comprising the steps of:
micronising the active substances in order to obtain these substances with average particle size in mentioned range,
micronising the excipients in order to obtain these excipients with average particle size in mentioned range,
putting the excipients with the coarser particle size into the mixing container,
putting the active substances with the finer particle size into the mixing container,
mixing the components of the medicament composition in the mixing container,
the medicament composition is filled to the blisters to make said medicament composition ready for use.

The medicament composition in accordance with the present invention can be used for the treatment of many respiratory diseases such as asthma, chronic obstructive pulmonary disorder (COPD) and allergic rhinitis. Accordingly, the respiratory diseases include but are not restricted to; allergic or non-allergic asthma in various phases, acute lung injury (ALI), acute respiratory distress syndrome (ARDS), exacerbation of airways hyperactivity, bronchiectasis, chronic obstructive pulmonary, airways or lung diseases (COPD, COAD or COLD) including emphysema and chronic bronchitis, pneumoconiosis, aluminosis, anthracosis, asbestosis, chalicosis, ptilosis, siderosis, silicosis, tabacosis and byssinosis. The treatment of said diseases may be prophylactic or symptomatic. In addition to this, the medicament composition in accordance with the present invention is used especially for the symptomatic treatment of asthma, allergic rhinitis and COPD.

The present invention is illustrated by examples given below, but it is not restricted to these examples. Parts given in examples represent the weights of ingredients.

### Example 1

In order to prepare the medicament composition of the present invention which is stored in a peelable blister strip to be used by the multi dose inhaler disclosed in GB2242134 or a similar one;
50 parts of salmeterol xinafoate with an average particle size in the range of 2 to 5 μm
100 parts of fluticasone propionate with an average particle size in the range of 2 to 5 μm
4000 parts of sodium cromoglycate with an average particle size in the range of 2 to 10 μm
10000 parts of sodium cromoglycate with an average particle size in the range of 80 to 100 μm are micronised in an air jet mill to obtain the substances mentioned above with average particle size in mentioned range and they are mixed in a mixing container.

In this example, the active substances may be extended to comprise their pharmaceutically acceptable salts, i.e. salmeterol xinofoate comprises all the pharmaceutically acceptable salts thereof, fluticasone propionate comprises all the pharmaceutically acceptable salts thereof, sodium cromoglycate comprises all the salts of cromoglicic acid and nedocromil. Also, optionally a pharmaceutically acceptable carrier can be added to the formulation and the example 1 is repeated with the amounts given below.

| | | | Amount of | Amount of |
|---|---|---|---|---|
| | | | A salt of | A salt of |
| | Amount of | Amount of | Cromoglicic | Cromoglicic |
| | Salmeterol | Fluticasone | Acid | Acid |
| | Xsinafoate | Propionate | (80-100 μm) | (2-10 μm) |
| | (part) | (part) | (part) | (part) |
| Example 2 | 50 | 100 | 2000 | 10000 |
| Example 3 | 50 | 250 | 4000 | 10000 |
| Example 4 | 50 | 250 | 2000 | 10000 |
| Example 5 | 50 | 500 | 4000 | 10000 |
| Example 6 | 50 | 500 | 2000 | 10000 |

1. A medicament composition comprising a combination of salmeterol or a pharmaceutically acceptable salt thereof and fluticasone or a pharmaceutically acceptable salt thereof used in the treatment of respiratory disorders by inhalation route characterized in that said medicament composition also contains a pharmaceutically acceptable salt of cromoglicic acid and/or nedocromil in an effective amount.

2. A medicament composition according to claim 1, wherein said medicament composition contains preferably salmeterol xinafoate.

3. A medicament composition according to claim 1, wherein said medicament composition contains preferably fluticasone propionate.

4. A medicament composition according to any one of the preceding claims, wherein said medicament composition contains a salt of cromoglicic acid, preferably sodium cromoglycate.

5. A medicament composition according to any one of the preceding claims, wherein said medicament composition contains a salt of nedocromil, preferably nedocromil sodium.

6. A medicament composition according to claim 1, wherein said medicament composition used locally for the treatment of respiratory diseases by inhalation route consists of micronized dry powder.

7. A medicament composition according to claim 6, wherein said medicament composition, which is in dry powder form, is administered directly to airways by using a dry powder inhalation device.

8. A dry powder medicament composition according to claim 6, wherein the active substance has an average particle size in the range of 1 to 5 μm.

9. A medicament composition according to any one of the preceding claims, wherein said medicament composition, that is in dry powder form, contains a β₂-agonist and a corticosteroid present in the amount of 1 to 1000 μg for each dose, a salt of cromoglicic acid and/or nedocromil present in the amount of 1 to 50 mg for each dose and a pharmaceutically suitable carrier present in a sufficient amount to adjust the total amount of each dose to the range of between 5 and 50 mg.

10. A medicament composition in dry powder form according to claim 9, wherein the ratio of the total amount of the combination of a β₂-agonist and a corticosteroid to the total amount of a salt of cromoglicic acid is in the range of 1:5 to 1:55000.

11. A medicament composition in dry powder form according to claim 1, wherein said composition optionally contains a pharmaceutically acceptable carrier.

12. A medicament composition according to claim 11, wherein said pharmaceutically acceptable carrier is selected from the group comprising monosaccharide, disaccharide, polysaccharide and oligosaccharide.

13. Use of a medicament composition containing the combination of a β₂-agonist and a corticosteroid with the salt of cromoglicic acid and/or nedocromil and optionally a pharmaceutically acceptable carrier by a dry powder inhaler, in which said medicament composition is stored in a peelable blister strip, for the treatment of respiratory diseases.

14. Use of the combination of β₂-agonist and corticosteroid with the salt of cromoglicic acid and/or nedocromil for the preparation of a medicament according to any one of the preceding claims for use in the treatment of inflammatory or obstructive respiratory diseases.

15. A method for the preparation of the medicament composition according to claim 1, which comprises the steps of:
micronising the active substances in order to obtain these substances with average particle size in mentioned range,
micronising the excipients in order to obtain these excipients with average particle size in mentioned range,
putting the excipients with the coarser particle size into the mixing container,
putting the active substances with the finer particle size into the mixing container,
mixing the components of the medicament composition in the mixing container,
the medicament composition is filled to the blisters to make said medicament composition ready for use.
